(19) United States
(12) Patent Application Publication
(10) Pub. No.: US 2020/0323735 A1
(43) Pub. Date: Oct. 15, 2020

(54) RELEASE MODULE GROUP DETERMINATION FOR DELIVERY OF A DRUG

(71) Applicant: Hewlett-Packard Development Company, L.P., Spring, TX (US)

(72) Inventors: Wei Huang, Palo Alto, CA (US); Gary J. Dispoto, Palo Alto, CA (US)

(73) Assignee: Hewlett-Packard Development Company, L.P., Spring, TX (US)

(21) Appl. No.: 16/755,604

(22) PCT Filed: Oct. 25, 2017
(86) PCT No.: PCT/US2017/058356

§ 371 Date: Apr. 13, 2020

(51) Int. Cl. A61J 3/07; G16H 70/40; G16H 50/20; G16H 20/13; G16H 50/70; G05B 15/02

(52) U.S. Cl. CPC A61J 3/07; G16H 70/40; G05B 15/02; G16H 20/13; G16H 50/70; G16H 50/20

(57) ABSTRACT

According to examples, an apparatus may include a memory on which is stored instructions that may cause a processor to receive an intended release profile for a drug, the intended release profile including a certain release rate of the drug over time following ingestion of the drug. The instructions may also cause the processor to determine a group of release modules that contain the drug, the determined group of release modules having release profiles for the drug that together meet or approximate the intended release profile for delivery of the drug, and at least two of the release modules in the group having different release profiles for the with respect to each other.

### BACKGROUND

Pharmaceutical dosage forms may be fabricated to contain a single type of drug or multiple types of drugs. The pharmaceutical dosage forms may also be fabricated to have a variety of drug release profiles, including an immediate release profile, a sustained release profile, and a delayed release profile. The pharmaceutical dosage forms may further be fabricated to have other types of drug release profiles in which drugs may be released at a timed release or at a pulsed release. Various techniques may be employed to fabricate the pharmaceutical dosage forms including, three-dimensional printing and other techniques.

### BRIEF DESCRIPTION OF THE DRAWINGS

Features of the present disclosure are illustrated by way of example and not limited in the following figure(s), in which like numerals indicate like elements, in which:

FIG. 1 shows a block diagram of an example apparatus that may determine a group of release modules having release profiles that together meet or approximate an intended release profile for delivery of a drug into a user;

FIGS. 2A-2C, respectively, depict graphs of release profiles of three example release modules and FIG. 2D shows a graph of an example combined release profile;

FIGS. 3A-3C, respectively, show perspective views of three example release modules;

FIG. 4 shows a diagram of an example process in which a group of the example release modules may be assembled into a dosage form;

FIG. 5A shows a block diagram of an example release module assembly system that may assemble a group of release modules into a dosage form;

FIG. 5B shows a diagram of an example 3D fabrication system that may fabricate a release module having a particular release profile and/or an ingestible dosage form having a particular release profile;

FIGS. 6 and 8, respectively show flow diagrams of example methods for determining a combination of tablets having a combined release profile that meets or is approximated to meet an intended release profile for administration of a drug into a user; and

FIG. 7A depicts a graph of an example intended or designed release profile of a drug over time, FIGS. 7B-7H, respectively, depict graphs of example release profiles of respective tablets, and FIG. 7I depicts a graph of example combined release profile of the release profiles depicted in FIGS. 7B-7H.

### DETAILED DESCRIPTION

Disclosed herein are apparatuses and methods for determining a group of release modules that contain a drug, in which the determined group of release modules may have release profiles for the drug that together meet or approximate an intended release profile for the drug. As discussed herein, at least some of the release modules may be fabricated to have different release profiles of the drug with respect to each other. For instance, one of the release modules may have a release profile in which the drug is delivered steadily for a duration of time, while another one of the release modules may have a release profile in which the drug is delivered at a fast rate initially and then gradually decreased. The combination of these two release modules may result in a combined release profile that differs from the individual release profiles. The apparatuses and methods disclosed herein may include identifying a combination of the release modules that, for instance, includes release profiles that together best match or best approximate an intended or designed release profile for delivery of a drug or for delivery of multiple drugs.

The apparatuses and methods disclosed herein may also assemble the release modules in the determined group into an ingestible dosage form, such as an ingestible capsule formed of two halves that may be joined following insertion of the release modules into the capsule. In this regard, the release modules may be of sufficiently small size such that a relatively large number of the release modules, e.g., anywhere from around 100 to around 1000 release modules, may be inserted into a single ingestible dosage form. In other examples, the release modules may be sized such that one release module or a relatively small number of release modules may be inserted into a single ingestible dosage form. In addition, the release modules may be pre-fabricated and the pre-fabricated release modules may be stored in a plurality of bins such that the release modules may be dispensed from the bins in their determined amounts to assemble the ingestible dosage form. The release modules may thus be fabricated off-site from where the ingestible dosage form is assembled. In other examples, however, the release modules may be fabricated at the same site as where the ingestible dosage form is assembled. In any regard, the release modules may be formed through any suitable fabrication technique in which, for example, a drug or multiple drugs are inserted into a medium for the drug or drugs.

Through implementation of the apparatuses and methods disclosed herein, ingestible dosage forms, e.g., capsules containing drugs, meeting particular drug release profiles may be assembled on-demand. That is, for instance, when a user seeks to obtain an ingestible dosage form meeting the particular drug release profile, the release modules for that ingestible dosage form may not need to be fabricated and thus, the ingestible dosage form may be assembled in more quickly as the release modules may be pre-fabricated.

As used herein, a drug may be defined as a medicine, an active ingredient, or other substance that may have a physiological effect when ingested or otherwise introduced into a body. As also used herein, a drug may include a dietary supplement such as a vitamin.

Before continuing, it is noted that as used herein, the terms “includes” and “including” mean, but is not limited to, “includes” or “including” and “includes at least” or “including at least.” The term “based on” means “based on” and “based at least in part on.”

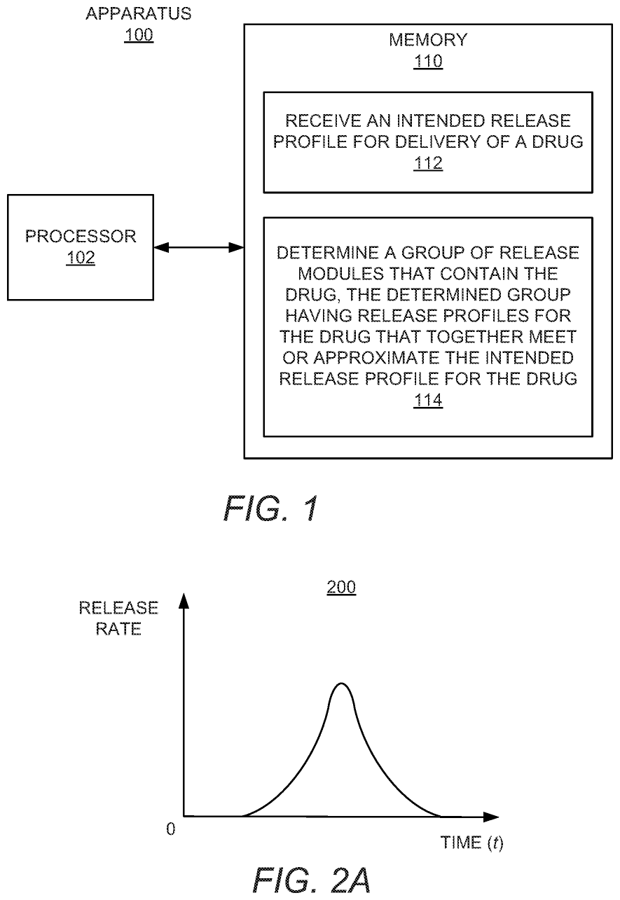

With reference first to FIG. 1, there is shown a block diagram of an example apparatus 100 that may determine a group of release modules having release profiles that together meet or approximate an intended release profile for delivery a drug into a user. It should be understood that the example apparatus 100 depicted in FIG. 1 may include additional features and that some of the features described herein may be removed and/or modified without departing from the scope of the apparatus 100.

Generally speaking, the apparatus 100 may be a computing device, a control device of a release module assembly system, or the like. As shown in FIG. 1, the apparatus 100 may include a processor 102 that may control operations of the apparatus 100. The processor 102 may be a semiconductor-based microprocessor, a central processing unit (CPU), an application specific integrated circuit (ASIC), a field-programmable gate array (FPGA), a core in a CPU, and/or other suitable hardware device.

The apparatus 100 may also include a memory 110 that may have stored thereon machine readable instructions 112 and 114 (which may also be termed computer readable instructions) that the processor 102 may execute. The memory 110 may be an electronic, magnetic, optical, or other physical storage device that contains or stores executable instructions. The memory 110 may be, for example, Random Access memory (RAM), an Electrically Erasable Programmable Read-Only Memory (EEPROM), a storage device, an optical disc, and the like. The memory 110, which may also be referred to as a computer readable storage medium, may be a non-transitory machine-readable storage medium, where the term “non-transitory” does not encompass transitory propagating signals.

The processor 102 may fetch, decode, and execute the instructions 112 to receive an intended release profile for delivery of a drug, in which the intended release profile may include a certain release rate of the drug following ingestion of the drug by a user. In other words, the intended release profile may include a desired rate at which the drug is to be released following ingestion of the drug by a user, e.g., a person, and immersion of the drug into a liquid. According to examples, a doctor or other medical personnel may determine the intended release profile for delivery of the drug to be administered to a patient, e.g., in the form of a prescription or other document, and the intended release profile may be entered into the apparatus 100 for processing. For instance, the medical personnel may determine the intended release profile for delivery of the drug for a patient based on the patient's symptoms and a treatment plan. Thus, for instance, the intended release profile for delivery of a drug (or equivalently, the intended release profile of the drug) to be administered to a patient may vary for different patients and/or conditions.

The processor 102 may fetch, decode, and execute the instructions 114 to determine a group of release modules that contain the drug, in which the determined group of release modules have release profiles for the drug that together meet or approximate the intended release profile for the drug. In some examples, the release modules in the determined group of release modules may have the same release profile. In these examples, the processor 102 may determine the group of release modules as the number of release modules that are to be included to meet the intended release profile for the drug. In addition or in other examples, at least two of the release modules in the group may have different release profiles for the drug with respect to each other. For instance, a first release module in the group may have an immediate release of the drug followed by a relatively lower release of the drug and a second release module in the group may have a steady release of the drug at a relatively constant rate. The combined release profiles of the first release module and the second release module in this example may thus approximate an intended release profile that includes a large initial release followed by a longer period of steady release of the drug.

In other implementations, instead of the memory 110, the apparatus 100 may include hardware logic blocks that may perform functions similar to the instructions 112 and 114. In yet other examples, the apparats 100 may include a combination of instructions and hardware logic blocks to implement or execute functions corresponding to the instructions 112 and 114. In any of these examples, the processor 102 may implement the hardware logic blocks and/or execute the instructions 112 and 114.

In determining the group of release modules, the processor 102 may determine both the type of release modules and the amount of the respective release modules to be included in the group. Thus, for instance, to approximate the intended release profile, the processor 102 may determine that a first number of first release modules having a first release profile may be included with a second number of second release modules having a second release profile. The processor 102 may thus determine the group to include release modules that together meet or approximate the intended release profile of the drug along with an intended dosage of the drug.

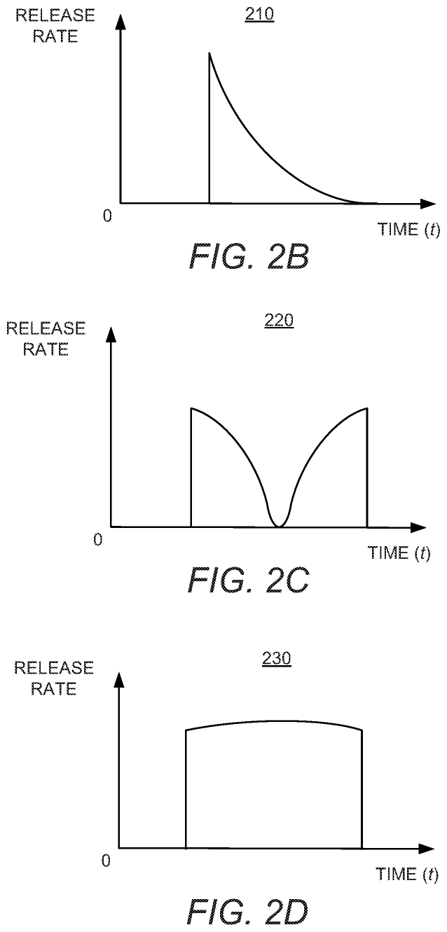

According to examples, the processor 102 may mathematically determine the release modules to be included in the group. For instance, the processor 102 may access a database containing the release profiles of the release modules. The release profiles of the release modules may be represented graphically as functions of drug release rates over time. Examples of release rates of a drug for three different release modules are depicted in the graphs 200, 210, and 220 respectively depicted in FIGS. 2A-2C. As shown in FIG. 2A, the graph 200 shows that a first release module may have a release profile in which the release rate of the drug is gradually increased over time, hits a peak rate, and then gradually decreases over time. As shown in FIG. 2B, the graph 210 shows that a second release module may have a release profile in which the release rate of the drug initially spikes and then gradually decreases over time. As shown in FIG. 2C, the graph 220 shows that a third release module may have a release profile in which the release rate of the drug initially spikes, gradually decreases, and then gradually increases before going to zero overtime.

The processor 102 may mathematically determine the release modules to be included in the group by determining combined release profiles resulting from combining the release profiles of different combinations of the release modules. That is, for instance, combining the release profile of the first release module (FIG. 2A) with the release profile of the third release module (FIG. 2C) may result in a combined release profile as shown in the graph 230 depicted in FIG. 2D. In this regard, the processor 102 may determine a combination of release modules that have release profiles, that when combined with each other, meet or approximately meet the intended release profile. A combined release profile may be considered to approximately meet the intended release profile when, for instance, a deviation between the combined release profile and the intended release profile is less than a predefined difference level. The predefined difference level may be based on various factors, such as, the potency of the drug, side effects of the drug, relative importance of meeting the intended release profile, etc.

In some examples, the processor 102 may receive a second intended release profile for delivery of a second drug. In these examples, the processor 102 may determine that the group may include a group of release modules having the second drug, that when combined, result in a combined release profile that meets or approximately meets the second intended release profile. The group of release modules having the second drug may overlap with some of the release modules previously determined to be in the group or may be separate release modules. In any regard, the release modules disclosed herein may include a single drug or may include multiple drugs. In instances in which the release modules include multiple drugs, the release profiles of the drugs in a release module may be the same as each other or may differ from each other. Thus, for instance, a first drug in the release module may have a first release profile and a second drug in the release module may have a different, second release profile. In some examples, the first drug may be dependent upon the second drug in a release module, e.g., the first drug may not function properly without the second drug. In these examples, the first drug and the second drug may be provided into the release module in a proportional manner.

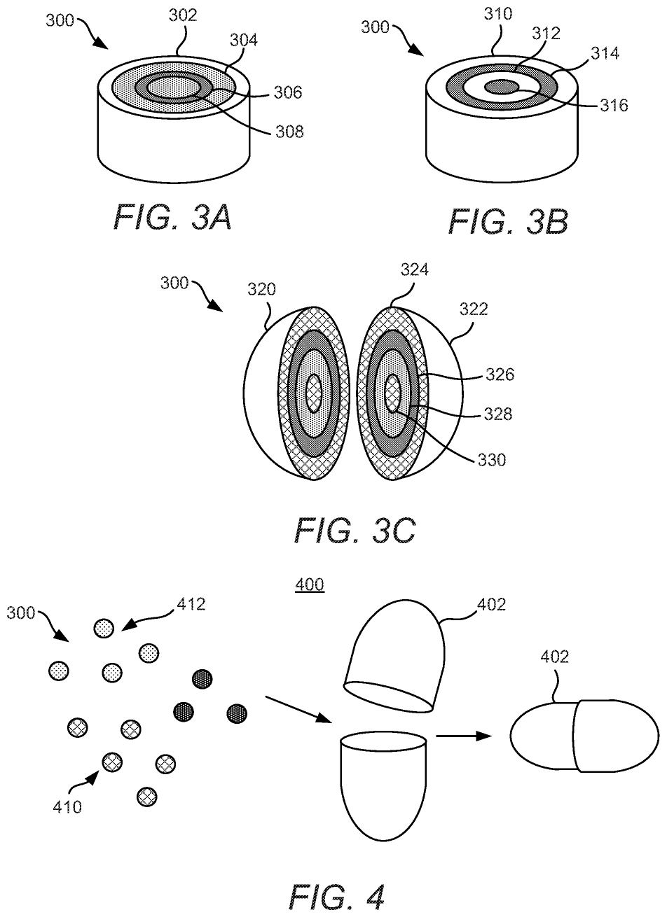

Turning now to FIGS. 3A-3C, there are shown perspective views of three example release modules 300. It should be understood that the example release modules 300 depicted in FIGS. 3A-3C may include additional features and that some of the features described herein may be removed and/or modified without departing from the scopes of the release modules 300.

Generally speaking, the release modules 300 depicted in FIGS. 3A-3C, which may equivalently be referenced as tablets, pellets, or the like, may have a different release profile with respect to each other. By way of example, the release profile for the release module 300 depicted in FIG. 3A may correspond to the release profile represented by the graph 200 depicted in FIG. 2A. That is, for instance, the release module 300 depicted in FIG. 3A may include an inactive section 302, a first drug section 304, a second drug section 306, and a third drug section 308. The first drug section 304 and the third drug section 308 may include a drug at a first concentration level and the second drug section 306 may include the drug at a second concentration level. As represented by the graph 200 in FIG. 2A, the second concentration level may be relatively higher than the first concentration level such that the release rate of the drug in the second drug section 306 may be relatively higher than the release rate of the drug in the first drug section 304 and the third drug section 308. The lengths of time during which the drugs are released may be controlled through control of the compositions of the drug sections 304-308 as well as the thicknesses of the drug sections 304-308.

When the release module 300 is immersed into a liquid, e.g., ingested into a body, the inactive section 302 of the release module 300 may begin to release. The inactive section 302 may be formed of an excipient that does not contain a drug or that may contain an inert substance and thus, when the inactive section 302 releases, a drug may not be released. The inactive section 302 may be formed to release at a predetermined rate and thus, the amount of time that it takes for the inactive section 302 to release may be controlled by varying the thickness of the inactive section 302. Thus, for instance, the timing at which the drug in the first drug section 304 begins to be released may be delayed by a certain amount of time by the thickness of the inactive section 302.

The release module 300 depicted in FIG. 3B may correspond to the release profile of the graph 220 depicted in FIG. 2C. That is, for instance, the release module 300 depicted in FIG. 3B may include a first inactive section 310, a second inactive section 312, a first drug section 314, and a second drug section 316. The first drug section 314 and the second drug section 316 may include a drug that is at a same concentration level. In addition, the release module 300 may include the second inactive section 312 to achieve the release profile depicted in FIG. 2C.

The release module 300 depicted in FIG. 3C may have a different shape as compared with the release modules 300 depicted in FIGS. 3A and 3B. That is, the release module 300 may have a spherical or an oval shape and each of the sections inside the release module 300 may also have spherical or oval shapes. The release module 300 has been depicted as being split into a first half 320 and a second half 322 to enable an interior of the release module 300 to be visible. As shown, the release module 300 may include a first drug section 324, a second drug section 326, a third drug section 328, and a fourth drug section 330. According to examples, the drug sections 324-330 may represent different concentrations of the same drug, such that the drug may be released at different release rates as the drug sections 324-330 are sequentially release. In addition, or in other examples, the drug sections 324-330 may represent at least two different drugs, such that the different drugs may be released as the drug sections 324-330 are sequentially released. In these examples, the different drugs may also be at different concentrations to thus enable the drugs to be released at different release rates.

The release module 300 has been discussed above as having various features for purposes of illustration and not of limitation. In this regard, it should be understood that the release module 300 may include other various features, such as additional types of drugs, concurrent release of multiple types of drugs, etc.

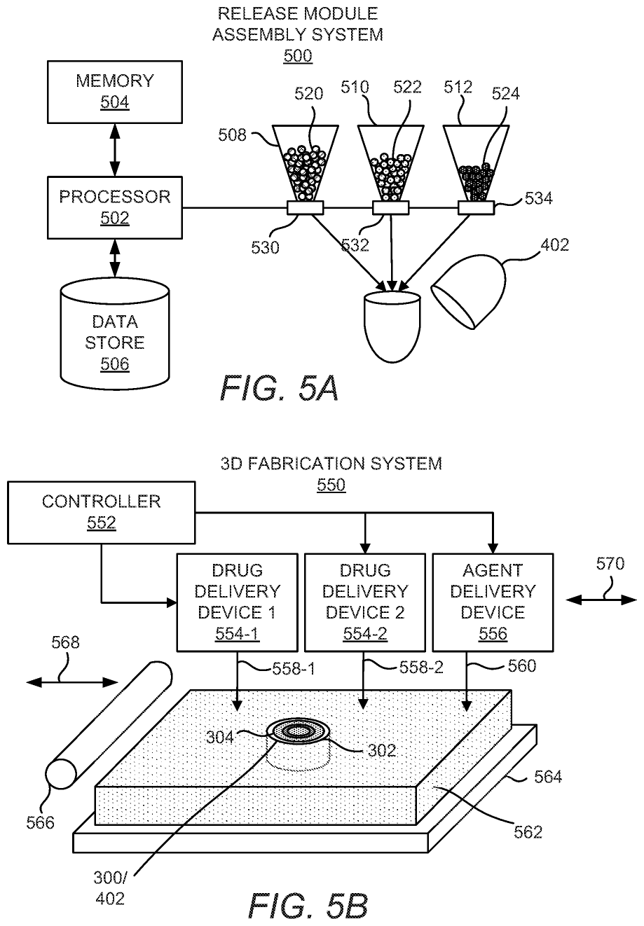

With reference now to FIG. 4, there is shown a diagram of an example process 400 in which a group of the release modules 300 may be assembled into a dosage form 402. The group of release modules 300 may include release modules 300 having a plurality of different release profiles. That is, for instance, one set 410 of release modules 300 may have a first release profile and another set 412 of release modules 300 may have a second release profile that differs from the first release profile. In addition, the processor 102 may have determined the group of release modules 300 such that the group of release modules 300 together meet or approximate an intended release profile for a drug or multiple drugs as discussed above with respect to FIG. 1.

As shown in FIG. 4, the group of release modules 300 may be assembled into the dosage form 402, which may be an ingestible capsule. That is, the group of release modules 300 may be deposited into the dosage form 402, which may be formed of two ingestible sections, and the two sections may be assembled to form an assembled dosage form 402. The dosage form 402 may be composed of a material or materials and may have a size suitable for humans to ingest the dosage form 402 and the release modules 300 may be sized to be smaller than the dosage form 402. In addition, the release modules 300 may be sufficiently small such that the dosage form 402 may house hundreds or thousands of the release modules 300. In some examples, a plurality of the dosage forms 402 may be assembled and placed into a container, e.g., a medicine bottle or other carrier for a patient.

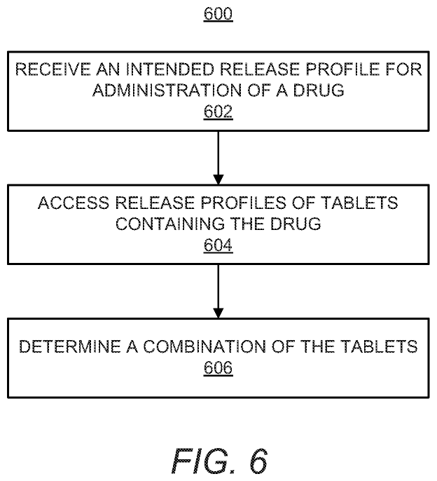

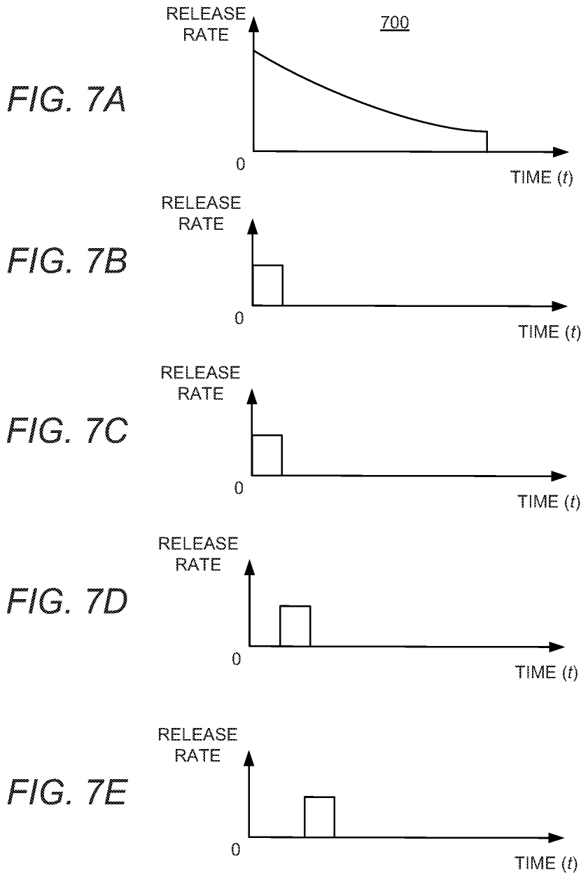

Turning now to FIG. 5A, there is shown a block diagram of an example release module assembly system 500 that may assemble a group of release modules 300 into a dosage form 402. It should be understood that the release module assembly system 500 depicted in FIG. 5A may include additional components and that some of the components described herein may be removed and/or modified without departing from a scope of the release module assembly system 500 disclosed herein.

As shown, the release module assembly system 500 may include a processor 502, a memory 504, and a data store 506. The processor 502 may be equivalent to the processor 102 and the memory 504 may be equivalent to the memory 110 depicted in FIG. 1. In this regard, the memory 504 may have stored thereon instructions 112, 114 that the processor 502 may execute to determine a group of release module 300 that together are to meet or approximately meet an intended release profile for a drug.

In addition, the data store 506 may have stored thereon the release profiles of a plurality of different release modules 300. The data store 506 may be an electronic, magnetic, optical, or other physical storage device that contains or stores executable instructions. The data store may be, for example, Random Access memory (RAM), an Electrically Erasable Programmable Read-Only Memory (EEPROM), a storage device, an optical disc, and the like. In some examples, the data store 506 and the memory 504 may be a single storage device.

The release module assembly system 500 may also include a plurality of bins 508-512, in which each of the bins 508-512 may house a different type of release module 300. The different types of release modules 300 may include release modules 300 having different release profiles of a drug with respect to each other. In addition, or in other examples, the different types of release modules 300 may include a first type of release module 300 including a first drug, a second type of release module 300 having a second drug, and so forth. In this regard, the type of release module 300 may refer to a type of drug that the release module 300 includes, a release profile of a type of drug that the release module 300 includes, combinations thereof, or the like.

As shown, the first bin 508 may house a first type of release module 520, the second bin 510 may house a second type of release module 522, and the third bin 512 may house a third type of release module 524. Each of the bins 508-512 may include a respective opening (not shown) through which the release modules 520-524 may be dispensed from the bins 508-512. As also shown, a respective dispense regulation device 530-534 may be attached to the bins to regulate the number and/or rate at which the release modules 520-524 may be dispensed from the respective bins 508-512. The dispense regulation devices 530-534 may include valves or other suitable devices that may regulate the dispensing of the release modules 520-524 from the bins 508-512.

According to examples, the dispense regulation devices 530-534 may be electronically operated. In these examples, the processor 502 may be electrically connected to the dispense regulation devices 530-534 and may individually control the dispense regulation devices 530-534. That is, for instance, the processor 502 may control the dispense regulation devices 530-534 to dispense the release modules 520-524 according to a determined group of release modules that are to meet or approximately meet an intended release profile for a drug or a plurality of drugs. The release modules 520-524 in the determined group may be dispensed into a dosage form 402, e.g., the release modules 520-524 may be dispensed into a chute that leads to the dosage form 402. In any regard, the dosage form 402, which may be a capsule having two sections, may be assembled to encapsulate the release modules dispensed into the dosage form 402.

According to examples, the release modules 520-524 may be pre-fabricated. That is, the release modules 520-524 may not be printed on-demand, e.g., as the release modules 520-524 are to be dispensed into the dosage form 402. Instead, the release modules 520-524 may be fabricated prior to a time at which the release modules 520-524 are to be dispensed. By way of example, the release module assembly system 500 may be located near patients, e.g., in a pharmacy, and the release modules 520-524 may be fabricated remotely from the release module assembly system 500.

In any regard, the release modules 520-524 may be fabricated in any of a variety of manners. For instance, the release modules 520-524 may be fabricated through any of a number of 3D printing techniques, an example of which is discussed below with respect to FIG. 5B. In addition, or in other examples, the release modules 520-524 may be fabricated through other techniques, e.g., conventional drug fabrication techniques. In any of these examples, the release modules 520-524 may be fabricated through use of excipients. The pre-fabricated release modules 520-524 may also be supplied into the respective bins 508-512 for storage and subsequent dispensing.

With reference now to FIG. 5B, there is shown a diagram of an example 3D fabrication system 550 that may fabricate a release module 300 having particular release profile and/or an ingestible dosage form 402 having a particular release profile. It should be understood that the 3D fabrication system 550 depicted in FIG. 5B may include additional components and that some of the components described herein may be removed and/or modified without departing from a scope of the 3D fabrication system 550 disclosed herein.

The 3D fabrication system 550 may include a controller 552, a first drug delivery device 554-1, a second drug delivery device 554-2, and an agent delivery device 556. The controller 552 may be a semiconductor-based microprocessor, a central processing unit (CPU), an application specific integrated circuit (ASIC), a field-programmable gate array (FPGA), and/or other suitable hardware device and may control operations of the 3D fabrication system 550. Particularly, for instance, the controller 552 may control the first drug delivery device 554-1 to selectively deposit a first drug, which is represented by the arrow 558-1, onto sets of particles 562 as part of a fabrication process to form a release module 300 or a dosage form 402. In some examples, the controller 552 may also control the second drug delivery device 554-2 to selectively deposit a second drug, which is represented by the arrow 558-2, onto sets of particles 562 as part of the fabrication process. The particles 562 may be particles of an excipient, e.g., an inactive substance that may serve as a medium for the first drug and/or the second drug. In addition, the particles 526 may be in a powder or a powder-like form.

The controller 552 may control the first drug delivery device 554-1 to selectively deposit the first drug 558-1 to place a controlled amount of the first drug 558-1, e.g., an intended dosage of the first drug 558-1, in the fabricated release module 300/dosage form 402. The controller 552 may also control the first drug delivery device 554-1 to selectively deposit the first drug 558-1 to place a controlled amount of the first drug 558-1 at particular locations of the release module 300/dosage form 402 to control the release profile, e.g., the release rate, of the first drug 558-1 from the release module 300/dosage form 402. That is, the controller 552 may identify an intended release profile and/or dosage for the first drug 558-1 in the release module 300/dosage form 402 and may control the first drug delivery device 554-1 to deliver the first drug 558 to fabricate a release module 300/dosage form 402 that may meet the intended release profile and/or dosage. Similarly, and in some examples, the controller 552 may identify an intended release profile and/or dosage for the second drug 558-2 in the release module 300/dosage form 402 and may control the second drug delivery device 554-2 to fabricate a release module 300/dosage form 402 that may meet the intended release profile and/or dosage. In any regard, the first drug 558-1 may differ from the second drug 558-2 such that, for instance, the 3D fabrication system 550 may be implemented to fabricate release modules 300 and/or dosage forms 402 having various combinations of drugs. In

The controller 552 may further control the agent delivery device 556 to selectively deliver an agent, which is represented by the arrow 560, onto the particles 562. The agent 560 may be a binding agent that may bind the particles 562 on which the agent 560 has been delivered. The agent 560 may be an inactive substance and may function to hold the particles 562 forming the release module 300/dosage form 402 together following deposition of the agent 560 onto the particles 562. The agent 560 may be activated through, for instance, contact with the particles 562, exposure to air, exposure to light, exposure to heat, or the like. In some implementations, the 3D fabrication system 550 may also include an activating device (not shown) that may be implemented to activate the agent 560. In addition, following activation of the agent 560, the agent 560 may dissolve in the presence of a liquid at a predetermined rate to thus enable the first drug 558-1 to be released, for instance, after the release module 300/dosage form 402 has been ingested.

In some examples, the controller 552 may control the agent delivery device 556 to deliver the agent 560 onto the particles 562 that are to form the release module 300/dosage form 402. In these examples, the controller 552 may control the first drug delivery device 554-1 to deliver the first drug 558-1 on at least some of the particles 502 on which the agent 560 is delivered. In addition, the particles 562 upon which the first drug 558-1 has not been deposited may be an inactive section 302 of a release module 300/dosage form 402 and the particles 562 upon which the first drug 558-1 has been deposited may be a drug section 304 of the release module 300/dosage form 402.

In some examples, the controller 552 may control the agent delivery device 556 to deliver the agent 560 onto some of the particles 562 that are to form the release module 300/dosage form 402. For instance, the controller 552 may control the first drug delivery device 554-1 to deliver the first drug 558-1 onto a first subset of those particles and may control the agent delivery device 556 to deliver the agent 560 onto another subset of those particles. As such, the first drug 558-1 and the agent 560 may be deposited onto particles 562 located at different areas. In these examples, the first drug 558-1 may include a binding agent and may bind the particles 562 upon which the first drug 558-1 has been deposited. The binding agent in the first drug 558-1 may be the same as the agent 560. The second drug 558-2 may also include a similar binding agent.

The first drug 558-1, the second drug 558-2, and the agent 560 may be respective liquids and may each be deposited in the form of droplets. In one regard, the controller 552 may have relatively fine-grain control over the locations at which each of the first drug 558-1, the second drug 558-2, and the agent 560 may be deposited.

The 3D fabrication system 550 may also include a build platform 564, which may be in a build chamber (which may define a build envelope) within which release modules 300/dosage forms 402 may be fabricated from particles 562 provided in respective layers on the build platform 564. Particularly, the build platform 564 may be provided in a build chamber and may be moved downward as portions of a release module 300/dosage form 402 are fabricated in the successive layers of particles 562. Although not shown, the particles 562 may be supplied between a recoater 566 and the build platform 564 and the recoater 566 may be moved in a direction represented by the arrow 560 across the build platform 564 to spread the particles 562 into a layer. In addition, the first drug delivery device 554-1, the second drug delivery device 554-2, and the agent delivery device 556 may be moved across the build platform 564 as indicated by the arrow 570 to deliver the first drug 558-1 and/or the second drug 558-2 and to bind together particles 562 in selected areas of layers of the particles 562.

For instance, the first drug delivery device 554-1, the second drug delivery device 554-2, and the agent delivery device 556 may be supported on a carriage that is to move in the directions 570. In some examples, the recoater 566 may be provided on the same carriage. In other examples, first drug delivery device 554-1, the second drug delivery device 554-2, and the agent delivery device 556 may be supported on a plurality of carriages such that the first drug delivery device 554-1 and the second drug delivery device 554-2, and the agent delivery device 556 may be moved separately with respect to each other. In any regard, following formation of a layer of particles 562 and a portion of a release module 300/dosage form 402, the recoater 566 may be implemented to form another layer and this process may be repeated to fabricate the release module 300/dosage form 402.

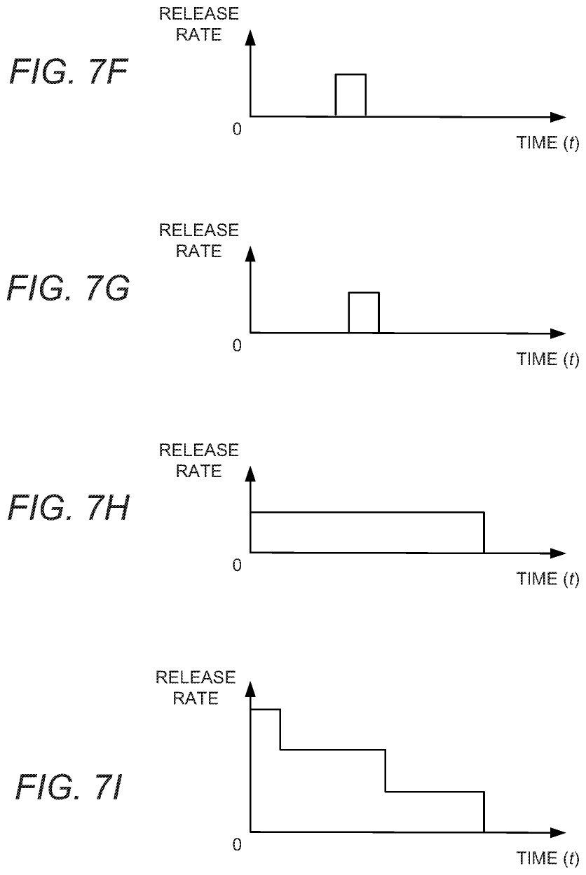

Various manners in which the processor 102, 502 may operate are discussed in greater detail with respect to the method 600 depicted in FIG. 6. Particularly, FIG. 6 depicts a flow diagram of an example method 600 for determining a combination of tablets 300 having a combined release profile that meets or is approximated to meet an intended release profile for administration of a drug into a user, e.g., a person. It should be understood that the method 600 depicted in FIG. 6 may include additional operations and that some of the operations described therein may be removed and/or modified without departing from scope of the method 600. The description of the method 600 is made with reference to the features depicted in FIGS. 1-5 for purposes of illustration.

At block 602, the processor 102, 502 may receive an intended release profile for a drug, in which the intended release profile may include a certain release rate over time of the drug. As discussed herein, a physician or other medical personnel may identify the intended release profile for the drug, in which the intended release profile may be for a particular user, e.g., patient. In addition, the intended release profile may be included in a prescription for the particular user and the processor 102, 502 may use the prescription to determine how the intended release profile may be met.

At block 604, the processor 102, 502 may access release profiles of a plurality of tablets 300, which may equivalently be termed release modules 300, containing the drug. As discussed herein, some of the tablets 300 may have different release profiles for the drug with respect to each other following immersion of the tablets 300 in a liquid. In any regard, the release profiles of the tablets 300 may be stored in a data store 506 and the processor 102, 502 may access the release profiles from the data store.

At block 606, the processor 102, 502 may determine a combination of the tablets 300 having a combined release profile that meets or is approximated to meet the intended release profile from the accessed release profiles of the tablets 300. As discussed herein, the processor 102, 502 may determine the combination of the tablets 300 using a mathematical approach on the accessed release profiles of the tablets.

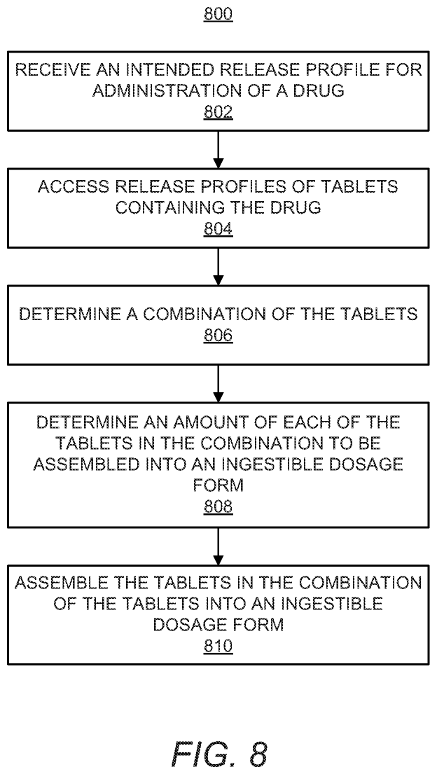

An example approach to determine the combination of the tablets 300 approximated to have the combined release profile is described with respect to a plurality of example drug release profile graphs shown in FIGS. 7A-7I. Particularly, FIG. 7A depicts a graph 700 of an example intended or designed release profile of a drug over time. FIGS. 7B-7H depict release profiles of respective release tablets 300 that the processor 102, 502 may determine to be combined to approximate the intended release profile of the drug. In addition or in other examples, each of the release profiles shown in FIGS. 7B-7H may correspond to a particular number of tablets 300. That is, for instance, FIG. 7B may depict a combined release profile of a particular number of tablets 300 of a first type, e.g., 100 or more tablets 300.

As shown in FIGS. 7B-7H, the determined combination may include a plurality of tablets 300 having the same release profile as well as tablets 300 having different release profiles. The combination of the tablets 300 having the release profiles shown in FIGS. 7B-7H may result in a combined release profile as shown in FIG. 7I. Particularly, FIG. 7I shows that the combined release profile may not exactly match the intended release profile shown in FIG. 7A, but instead, may include a stepped release profile that is approximately equivalent to the intended release profile. In one regard, the processor 102, 502 may determine a combination of tablets 300 having release profiles of the drug that together approximate the intended release profile of the drug.

With reference now to FIG. 8, there is shown a flow diagram of another example method 800 for determining a combination of release modules 300 having a combined release profile that meets or is approximated to meet an intended release profile for administration of a drug. It should be understood that the method 800 depicted in FIG. 8 may include additional operations and that some of the operations described therein may be removed and/or modified without departing from scope of the method 800. The description of the method 800 is made with reference to the features depicted in FIGS. 1-5 for purposes of illustration.

The method 800 is similar to the method 600 depicted in FIG. 6, but may include additional operations as compared to the method 600. For instance, blocks 802-806 may respectively be equivalent to blocks 602-606 in FIG. 6. As such, blocks 802-806 are not described in detail herein. In addition, following block 806 or as part of block 806, the processor 102, 502 may determine an amount of each of the tablets 300 in the determined combination of the tablets 300 to be assembled into an ingestible dosage form 402, as indicated at block 808. Thus, for instance, the processor 102, 502 may determine a first number of a first type of tablet 300 included in the determined combination to be included in the ingestible dosage form 402, a second number of the second type of tablet 300 included in the determined combination to be included in the adjustable dosage form 402, etc. As discussed herein, the combined release profile of the determined combination of tablets 300 may include the combined release profile combinations of each of a plurality of the different types of tablets 300.

At block 810, the processor 102, 502 may assemble the tablets 300 in the determine combination of the tablets 302 into an ingestible dosage form 402. For instance, the processor 102, 502 may control dispense regulation devices 530-534 to selectively expel the tablets 520-524 from respective bins 508-512 in the amounts determined at block 808. The expelled tablets 520-534 may be assembled into an ingestible dosage form 402, which may be in the form of a capsule as shown, for instance, in FIGS. 4 and 5 and discussed above.

According to examples, a plurality of digestible dosage forms 402 may be assembled at block 810 and the assembled digestible dosage forms 402 may be supplied into a medicine bottle or other support structure.

Although particular reference has been made herein to the combination of tablets 300 (or equivalently, release modules 300) that meet or approximately meet the intended release profile for a single drug, it should be understood that the methods 600, 800 disclosed herein may also be implemented to determine combinations of tablets 300 that need or approximately needs the intended release profiles for multiple drugs. Thus, for instance, the processor 102, 502 may receive an intended release profile for a second drug, in which the intended release profile for the second drug may include a certain release rate over time of the second drug following ingestion of the second drug. The processor 102, 502 may also identify tablets 300 that contain the second drug, in which at least two of the identified tablets 300 have different release profiles for the second drug with respect to each other. The processor 102, 502 may further determine a group of the identified tablets 300 containing the second drug that together approximate the intended release profile for the second drug. In addition, The tablets 300 in the determined group of the identified tablets 300 may be assembled into an ingestible dosage form 402. An ingestible dosage form 402 containing tablets 300 having release profiles for multiple drugs that approximate the intended release profile of the multiple drugs may thus be assembled.

Some or all of the operations set forth in the methods 600 and 800 may be included as utilities, programs, or subprograms, in any desired computer accessible medium. In addition, the methods 600 and 800 may be embodied by computer programs, which may exist in a variety of forms both active and inactive. For example, they may exist as machine readable instructions, including source code, object code, executable code or other formats. Any of the above may be embodied on a non-transitory computer readable storage medium.

Examples of non-transitory computer readable storage media include computer system RAM, ROM, EPROM, EEPROM, and magnetic or optical disks or tapes. It is therefore to be understood that any electronic device capable of executing the above-described functions may perform those functions enumerated above.

Although described specifically throughout the entirety of the instant disclosure, representative examples of the present disclosure have utility over a wide range of applications, and the above discussion is not intended and should not be construed to be limiting, but is offered as an illustrative discussion of aspects of the disclosure.

What has been described and illustrated herein is an example of the disclosure along with some of its variations. The terms, descriptions and figures used herein are set forth by way of illustration only and are not meant as limitations. Many variations are possible within the spirit and scope of the disclosure, which is intended to be defined by the following claims—and their equivalents—in which all terms are meant in their broadest reasonable sense unless otherwise indicated.

What is claimed is:

1. An apparatus comprising:
a processor; and
a memory on which is stored machine readable instructions that when executed by the processor, cause the processor to:
receive an intended release profile for delivery of a drug, the intended release profile including a certain release rate of the drug over time following ingestion of the drug; and
determine a group of release modules that contain the drug, the determined group of release modules having release profiles for the drug that together meet or approximate the intended release profile for delivery of the drug, and at least two of the release modules in the group having different release profiles for the drug with respect to each other.

2. The apparatus of claim 1, further comprising:
a plurality of bins, each of the plurality of bins housing release modules having different release profiles with respect to each other; and
dispense regulation devices attached to respective openings in the plurality of bins.

3. The apparatus of claim 2, wherein the instructions are further to cause the processor to:
control the dispense regulation devices to dispense the determined group of release modules from corresponding ones of the plurality of bins to assemble the determined group of release modules into an ingestible dosage form, the determined group of release modules including a determined number of each of the release modules in the determined group having different release profiles.

4. The apparatus of claim 3, wherein the ingestible dosage form comprises an ingestible capsule having a hollow core and the determined group of release modules are dispensed within the ingestible capsule.

5. The apparatus of claim 1, wherein the instructions are further to cause the processor to:
receive an intended release profile for delivery of a second drug, the intended release profile for delivery of the second drug including a certain release rate over time of the second drug following ingestion of the second drug;
identify release modules containing the second drug, at least two of the identified release modules having different release profiles for the second drug with respect to each other; and
determine a group of the identified release modules containing the second drug that together approximate the intended release profile for the second drug.

6. The apparatus of claim 5, wherein the plurality of release modules are housed in a plurality of bins, and wherein the instructions are further to cause the processor to:
control dispense regulation devices on the plurality of bins to dispense the determined group of release modules and the determined group of identified release modules into an ingestible dosage form.

7. The apparatus of claim 1, further comprising:
a data store on which is stored the release profiles of the release modules, wherein the instructions are further to cause the processor to access the data store to determine the group of the group of release modules that together approximate the intended release profile for the drug.

8. A computer-implemented method comprising:
receiving, by a processor, an intended release profile for administration of a drug, the intended release profile including a certain release rate over time of the drug;
accessing, by the processor, release profiles of tablets containing the drug, some of the tablets having different release profiles for the drug with respect to each other following immersion of the tablets in a liquid; and
determining, by the processor and from the accessed release profiles of the tablets, a combination of the tablets having a combined release profile that meets or is approximated to meet the intended release profile.

9. The method of claim 8, further comprising:
assembling the tablets in the determined combination of the tablets into an ingestible dosage form.

10. The method of claim 9, wherein the tablets are stored in separate bins and wherein assembling the determined combination of the tablets further comprises dispensing the determined combination of the tablets into the digestible dosage form from the separate bins.

11. The method of claim 9, further comprising:
determining an amount of each of the tablets in the determined combination to be assembled into the ingestible dosage form; and
dispensing the determined amount of each of the tablets in the determined combination into the ingestible dosage form.

12. The method of claim 8, further comprising:
receiving an intended release profile for administration of a second drug, the intended release profile for the second drug including a certain release rate over time of the second drug following ingestion of the second drug;
identifying tablets containing the second drug, at least two of the identified tablets have different release profiles for the second drug with respect to each other;
determining a group of the identified tablets containing the second drug that together approximate the intended release profile for the second drug; and
assembling the tablets in the determined group of the identified tablets into an ingestible dosage form.

13. The method of claim 8, wherein determining the combination of the tablets further comprises mathematically identifying the combination of the tablets having combined release profiles that are approximated to meet the intended release profile.

14. A non-transitory computer readable medium on which is stored machine-readable instructions but when executed by a processor cause the processor to:
receive an intended release profile for a drug to be administered to a user, the intended release profile including a certain release rate over time of the drug;
access release profiles of release modules containing the drug, some of the release modules having different release profiles for the drug following immersion of the release modules with respect to each other;
determine, from the accessed release profiles of the release modules, a group of the release modules having a combined release profile that meets or is approximated to meet the intended release profile; and
cause the determined group of the release modules to be dispensed into an ingestible dosage form.

15. The non-transitory computer readable medium of claim 14, wherein the instructions are further to cause the processor to:
determine the group of release modules through a mathematical approximation using the release profiles of the drug corresponding to the release modules.
